(19) United States
(12) Patent Application Publication
(10) Pub. No.: US 2005/0056280 A1
(43) Pub. Date: Mar. 17, 2005

(54) Receptacle for an aerosolizable pharmaceutical formulation

(75) Inventors: William W. Alston, San Jose, CA (US); Marc S. Gordon, Sunnyvale, CA (US)

Correspondence Address: NEKTAR THERAPEUTICS, 150 INDUSTRIAL ROAD, SAN CARLOS, CA, 94070, US

(73) Assignee: Nektar Therapeutics, San Carlos, CA (US)

(21) Appl. No.: 10/729,832

(22) Filed: Dec. 5, 2003

Related U.S. Application Data
(60) Provisional application No. 60/437,254, filed on Dec. 31, 2002.

(51) Int. Cl. A61M 15/00; A61M 16/00

(52) U.S. Cl. 128/203.21

(57) ABSTRACT

A method of aerosolizing a pharmaceutical formulation comprises providing an aerosolization device comprising an aerosolization chamber and providing a receptacle containing a pharmaceutical formulation. The receptacle comprises a wall having a weakened portion that opens when a force is applied. By applying a force to the receptacle to create an opening at the weakened portion, the pharmaceutical formulation in the receptacle is exposed so that it may be aerosolized for delivery to a patient's respiratory tract. In one version, the receptacle is opened when a blunt force is applied.

This application claims the benefit U.S. Provisional Patent Application Ser. No. 60/437,254 filed on Dec. 31, 2002, which is incorporated herein by reference in its entirety.

### BACKGROUND

The need for effective therapeutic treatment of patients has resulted in the development of a variety of pharmaceutical formulation delivery techniques. One traditional technique involves the oral delivery of a pharmaceutical formulation in the form of a pill, capsule, elixir, or the like. However, oral delivery can in some cases be undesirable. For example, many pharmaceutical formulations may be degraded in the digestive tract before they can be effectively absorbed by the body. Inhaleable drug delivery, where an aerosolized pharmaceutical formulation is orally or nasally inhaled by a patient to deliver the formulation to the patient's respiratory tract, has proven to be a particularly effective and/or desirable alternative. For example, in one inhalation technique, an aerosolized pharmaceutical formulation provides local therapeutic relief to a portion of the respiratory tract, such as the lungs, to treat diseases such as asthma, emphysema, and cystic fibrosis. In another inhalation technique, a pharmaceutical formulation is delivered deep within a patient's lungs where it may be absorbed into the blood stream. Many types of inhalation devices exist including devices that aerosolize a dry powder pharmaceutical formulation.

One type of inhalation device aerosolizes a pharmaceutical formulation that is stored in a capsule. For example, a dose or a portion of a dose of a dry powder pharmaceutical formulation may be stored in a capsule, and the capsule may be inserted into an aerosolization device which is capable of aerosolizing the pharmaceutical formulation. After being inserted into the aerosolization device, the capsule is opened to expose the pharmaceutical formulation. The opening of the capsule may be performed, for example, by puncturing or tearing the capsule. When the capsule is properly opened and when aerosolization energy is supplied, the pharmaceutical formulation is aerosolized so that it may be inhaled by the user and a dose or portion of a dose of the aerosolized pharmaceutical formulation may be delivered to the user's respiratory tract.

However, improper use of the aerosolization device may result in the delivery of less than the desired amount of the pharmaceutical formulation. For example, if a capsule is not properly or completely opened before the aerosolization process, the amount of pharmaceutical formulation being aerosolized may be reduced or the flow of the aerosolized pharmaceutical formulation may not be of sufficiently high quality to deliver a desirable amount to the user. This improper opening is particularly prevalent when a user is unable or unwilling to visually inspect the opening of the capsule. The user may then unknowingly inhale less than a desired amount of the pharmaceutical formulation. In addition, sharpened elements for creating the opening in the capsule may produce inconsistent openings into the capsule which can result in inconsistent delivery of aerosolized medicament.

Therefore, it is desirable to be able to provide a receptacle for an aerosolizable pharmaceutical formulation that is readily and consistently openable. It is further desirable to be able to provide such opening without the use of sharpened elements.

### SUMMARY

The present invention satisfies these needs. In one aspect of the invention, a receptacle is openable without using a sharpened tip.

In another aspect of the invention, an aerosolization system comprises an aerosolization device comprising a chamber adapted to receive a receptacle. The aerosolization system also comprises a receptacle containing a pharmaceutical formulation, the receptacle comprising a wall having a weakened portion that opens when a force is applied. An opening into the receptacle may be created at the weakened portion before, during, or after insertion of the receptacle into the chamber by applying a force to the receptacle.

In another aspect of the invention, a method of aerosolizing a pharmaceutical formulation comprises providing an aerosolization device comprising a chamber; providing a receptacle containing a pharmaceutical formulation, the receptacle comprising a wall having a weakened portion that opens when a force is applied; applying a force to the receptacle to create an opening at the weakened portion; before, during, or after applying the force to the receptacle, inserting the receptacle into the chamber; and aerosolizing the pharmaceutical formulation in the chamber.

In another aspect of the invention, a receptacle is provided for use in an aerosolization device comprising a chamber adapted to receive the receptacle. The receptacle comprises a wall having a weakened portion that opens when a force is applied and a pharmaceutical formulation within the wall, whereby an opening may be created at the weakened portion before, during, or after insertion of the receptacle into the chamber by applying a force to the receptacle.

### DRAWINGS

These features, aspects, and advantages of the present invention will become better understood with regard to the following description, appended claims, and accompanying drawings which illustrate exemplary features of the invention. However, it is to be understood that each of the features can be used in the invention in general, not merely in the context of the particular drawings, and the invention includes any combination of these features, where:

FIG. 1A is a schematic sectional side view of an aerosolization apparatus and receptacle in an initial position;

FIG. 1B is a schematic sectional side view of the aerosolization apparatus and receptacle shown in FIG. 1A at the beginning a receptacle opening process;

FIG. 1C is a schematic sectional side view of the aerosolization apparatus and receptacle shown in FIG. 1A during the a receptacle opening process;

FIG. 1D is a schematic sectional side view of the aerosolization apparatus and receptacle shown in FIG. 1A during the beginning of an aerosolization process;

FIG. 1E is a schematic sectional side view of the aerosolization apparatus and receptacle shown in FIG. 1A during the aerosolization process;

FIGS. 2A and 2B are schematic perspective views of a version of a receptacle according to the invention in an unopened and an opened condition, respectively;

FIGS. 3A through 3E are schematic sectional side views of a receptacle opening and aerosolization process using a receptacle according to the invention in another version of an aerosolization apparatus;

FIGS. 4A and 4B are schematic perspective views of another version of a receptacle according to the invention in an unopened and an opened condition, respectively;

FIGS. 5A through 5C are schematic sectional side views showing a receptacle according to the invention being opened by another version of an opening mechanism;

FIGS. 6A through 6C are schematic sectional side views showing a receptacle according to the invention being used in a version of an aerosolization apparatus which includes an opening mechanism with a flexible side wall;

FIGS. 7A and 7B are schematic perspective views of another version of a receptacle according to the invention in an unopened and an opened condition, respectively;

FIGS. 8A and 8B are schematic perspective views of another version of a receptacle according to the invention in an unopened and an opened condition, respectively;

FIG. 9A is a schematic perspective views of an elongated portion of another version of a receptacle according to the invention in an unopened condition;

FIG. 9B is a schematic sectional view of the elongated portion of FIG. 9A in a stressed and in an unstressed condition;

FIG. 10A is a schematic perspective views of an elongated portion of another version of a receptacle according to the invention in an unopened condition;

FIG. 10B is a schematic sectional view of the elongated portion of FIG. 10A in a stressed and in an unstressed condition.

### DESCRIPTION

The present invention relates to a receptacle for storing a pharmaceutical formulation. Although the process is illustrated in the context of storing an aerosolozable dry powder pharmaceutical formulation in a receptacle, the present invention can be used in other processes and should not be limited to the examples provided herein.

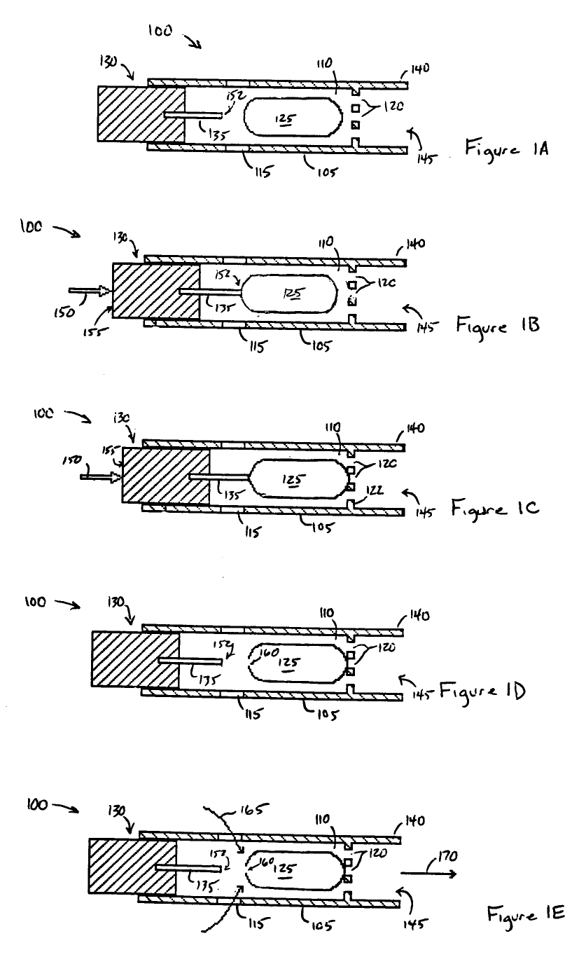

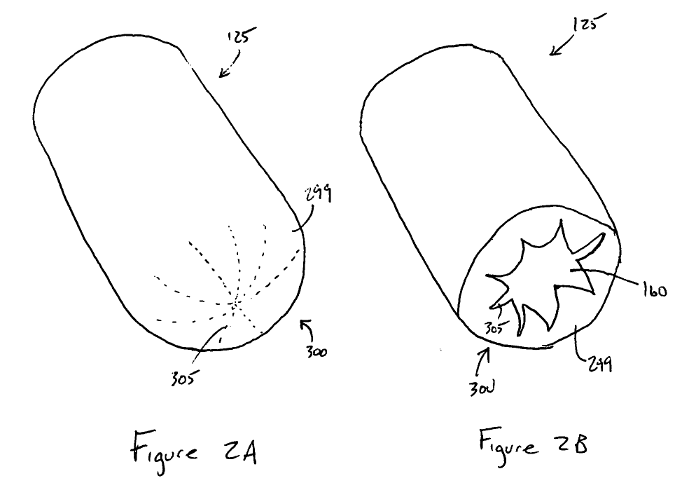

An aerosolization apparatus 100 and pharmaceutical formulation receptacle 125 according to the present invention is shown schematically in FIG. 1A. The aerosolization apparatus 100 comprises a housing 105 defining a chamber 110 having one or more air inlets 115 and one or more air outlets 120. The chamber 110 is sized to receive a receptacle 125 which contains an aerosolizable pharmaceutical formulation. An opening mechanism 130 comprises an opening member 135 that is moveable within the chamber 110. Near or adjacent the outlet 120 is an end section 140 that may be sized and shaped to be received in a user's mouth or nose so that the user may inhale through an opening 145 in the end section 140 that is in communication with the outlet 120.

The aerosolization apparatus 100 utilizes air flowing through the chamber 110 to aerosolize the pharmaceutical formulation in the receptacle 125. For example, FIGS. 1A through 1E illustrate the operation of a version of an aerosolization apparatus 100 where air flowing through the inlet 115 is used to aerosolize the pharmaceutical formulation and the aerosolized pharmaceutical formulation flows through the outlet 120 so that it may be delivered to the user through the opening 145 in the end section 140. The aerosolization apparatus 100 is shown in its initial condition in FIG. 1A. The receptacle 125 is positioned within the chamber 110 and the pharmaceutical formulation is contained within the receptacle 125.

To use the aerosolization apparatus 100, the pharmaceutical formulation in the receptacle 125 is exposed to allow it to be aerosolized. In the version of FIGS. 1A though 1E, the opening mechanism 130 is advanced within the chamber 110 by applying a force 150 to the opening mechanism 130. For example, a user may press against a surface 155 of the opening mechanism 130 to cause the opening mechanism 130 to slide within the housing 105 so that the opening member 135 contacts the receptacle 125 in the chamber 110, as shown in FIG. 1B. By continuing to apply the force 150, the opening member 135 is advanced to abut the forward wall 122 of the receptacle 125, as shown in FIG. 1C. The opening member may comprise one or more blunt tips 152 that contact the receptacle 125 in a manner that provides an opening into the receptacle 125. The opening mechanism 130 is then retracted to the position shown in FIG. 1D, leaving an opening 160 through the wall of the receptacle 125 to expose the pharmaceutical formulation in the receptacle 125.

Air or other gas then flows through an inlet 115, as shown by arrows 165 in FIG. 1E. The flow of air causes the pharmaceutical formulation to be aerosolized. When the user inhales 170 through the end section 140 the aerosolized pharmaceutical formulation is delivered to the user's respiratory tract. In one version, the air flow 165 may be caused by the user's inhalation 170. In another version, compressed air or other gas may be ejected into the inlet 115 to cause the aerosolizing air flow 165.

Proper creation of the opening 160 in the receptacle 125 allows for efficient and effective delivery of the aerosolized pharmaceutical formulation to the user. In contrast, improper creation of the opening 160 can lead to inefficient and less effective delivery of the medicament to a user. Accordingly, in one version, the receptacle 125 is designed to be at least partially self-opening when force is applied thereto. For example, as shown in FIG. 2A, the receptacle 125 may comprise a wall 299 having a weakened portion 300 that opens when a force is applied, such as a non-puncturing force. The weakened portion 300 comprises a region of the wall altered so as to fracture at a force less than would be necessary without the alteration. The alteration is added for the purpose of lessening the required fracture force and/or for the purpose of controlling the fracture. In one version, the weakened portion 300 comprises one or more scored or otherwise weakened lines 305. When a blunt force, such as a force from tip 152, is applied to the weakened portion 300, the lines 305 break and an opening 160 is created, as shown in FIG. 2B.

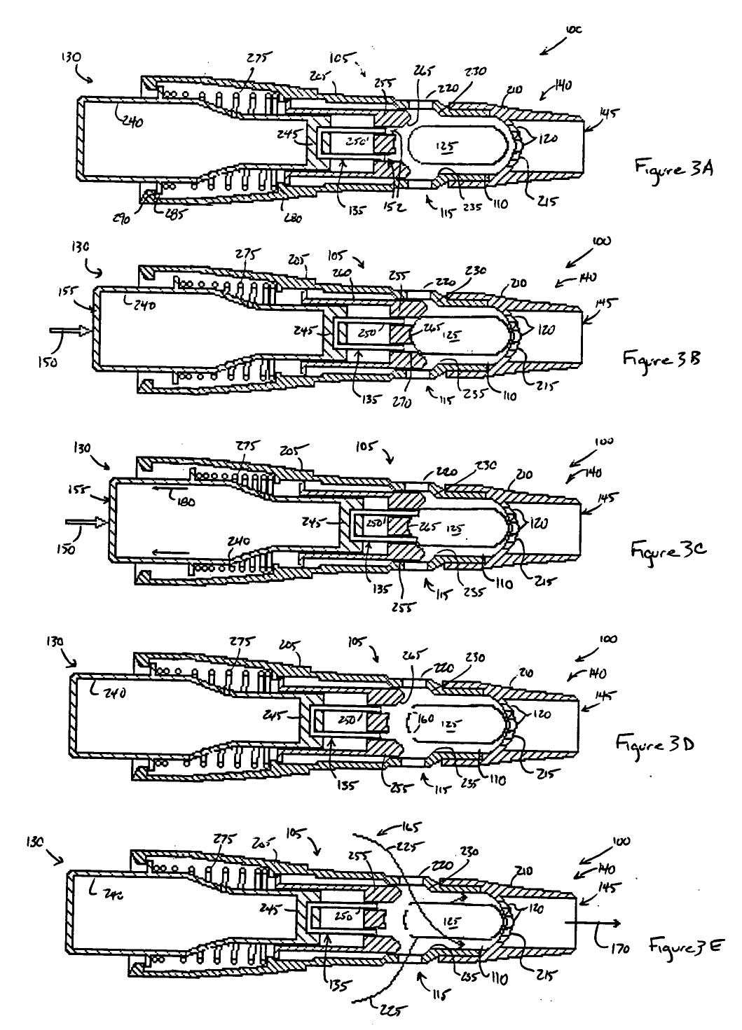

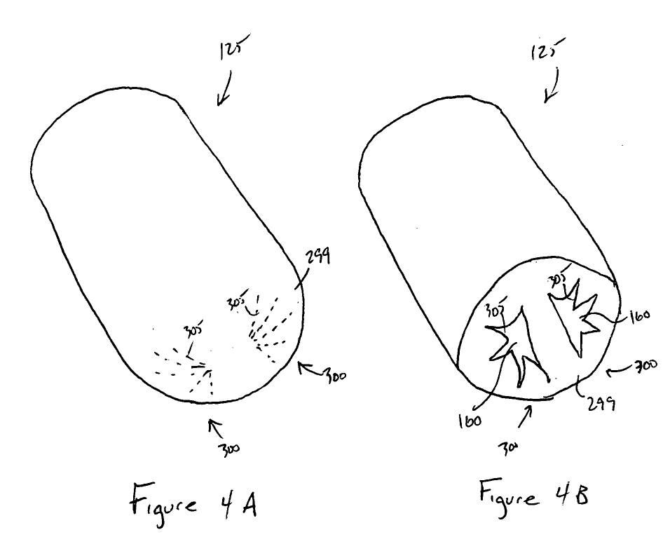

Another version of an aerosolization apparatus 100 comprising a blunt opening member 135 having a plurality of tips 152 for opening a receptacle 125 with a plurality of weakened portions 300 is shown in FIGS. 3A through 3E. In this version, the housing 105 of the aerosolization apparatus 100 comprises a body 205 and a removable endpiece 210. The endpiece 210 may be removed from the body 205 to insert a receptacle 125 in the chamber 110 which is formed when the body 205 and the endpiece 210 are connected together. The endpiece 210 comprises a partition 215 that blocks the forward end of the chamber 110, and the partition 215 has the one or more outlets 120 extending therethrough. An example of an aerosolization apparatus with a partition 215 and chamber 110 are described in U.S. Pat. No. 4,069,819 and in U.S. Pat. No. 4,995,385, both of which are incorporated herein by reference in their entireties. In such an arrangement, the chamber 110 comprises a longitudinal axis that lies generally in the inhalation direction, and the receptacle 125 is insertable lengthwise into the chamber 110 so that the receptacle's longitudinal axis may be parallel to the longitudinal axis of the chamber 110. In the version of FIGS. 3A through 3E, the chamber 110 is sized to receive a receptacle 125 containing a pharmaceutical formulation in a manner which allows the receptacle to move within the chamber 110. The inlets 115 comprise a plurality of tangentially oriented slots 220. When a user inhales 170 through the endpiece 210, outside air is caused to flow through the tangential slots 220 as shown by arrows 225 in FIG. 3E. This airflow 225 creates a swirling airflow within the chamber 110. The swirling airflow causes the receptacle 125 to contact the partition 215 and then to move within the chamber 110 in a manner that causes the pharmaceutical formulation to exit the receptacle 125 and become entrained within the swirling airflow. In one version, the receptacle 125 may rotate within the chamber 110 in a manner where the longitudinal axis of the receptacle, which may be a capsule, remains at an angle less than 80 degrees, and preferably less than 45 degrees from the longitudinal axis of the chamber. The movement of the receptacle 125 in the chamber 110 may be caused by the width of the chamber 110 being less than the length of the receptacle 125. In one specific version, the chamber 110 comprises a tapered section 230 that terminates at an edge 235. During the flow of swirling air in the chamber 110, the forward end of the receptacle 125 contacts and rests on the partition 215 and a sidewall of the receptacle 125 contacts the edge 235 and slides and/or rotates along the edge 235. This motion of the receptacle, which may be a capsule, is particularly effective in forcing a large amount of the pharmaceutical formulation through the plurality of openings 160 in the rear of the receptacle 125.

The plurality of openings 160 in the rear of the receptacle 125 in the version of FIGS. 3A through 3E are created by an opening mechanism 130 that is slidable within the body 205. The opening mechanism 130, shown in its rest position in FIG. 3A, comprises a plunger 240 attached at its forward end 245 to the opening member 135, which in the version shown is a U-shaped staple 250 having a plurality of blunt tips 152, such as the two tips shown in this version. The opening mechanism 130 further comprises a seating member 255 which contacts the plunger 240 and/or the opening member 135 and is slidable relative to the plunger 240 and the opening member 135. To create the openings 160 in the receptacle 125, the user applies a force 150 to the plunger 240, as shown in FIG. 3B, such as by pressing against the end surface 155 of the plunger 240 with the user's finger or thumb. The force 150 causes the plunger to slide within the body 205. A slight frictional contact between the plunger 240 the a rear section 260 of the seating member 255 causes the seating member 255 to also slide within the body 205 until a forward seating surface 265 of the seating member 255 contacts the receptacle 125, as shown in FIG. 3B. The forward seating surface 265, which may be shaped to generally match the shape of the receptacle 125, secures the receptacle 125 between the seating member 255 and the partition 215. The continued application of force 150 causes the plunger 240 and the opening member 135 to slide relative to the seating member 255, as shown in FIG. 3C, to advance the opening member 135 through openings 270 in the forward seating surface 265 and to the receptacle 125 to create the openings 160 as discussed above. Upon the removal of the force 150, a spring 275 or other biasing member urges the opening mechanism 130 back to its rest position. For example, the spring 275 may contact a shoulder 280 in the body 205 and press a flange 285 on the plunger 240 toward a rim 290 in the body 205. The frictional engagement between the plunger 240 and the seating member 255 also returns the seating member 255 to its retracted position when the plunger is returned to its retracted position.

A receptacle 125 that may be used with the aerosolization apparatus 100 of FIGS. 3A through 3E is shown in FIGS. 4A and 4B. The receptacle 125 of FIG. 4A comprises a plurality of weakened portions 300, each of which is contacted by a blunt tip 152 of the aerosolization apparatus 100 to create the plurality of openings 160, as shown in FIG. 4B.

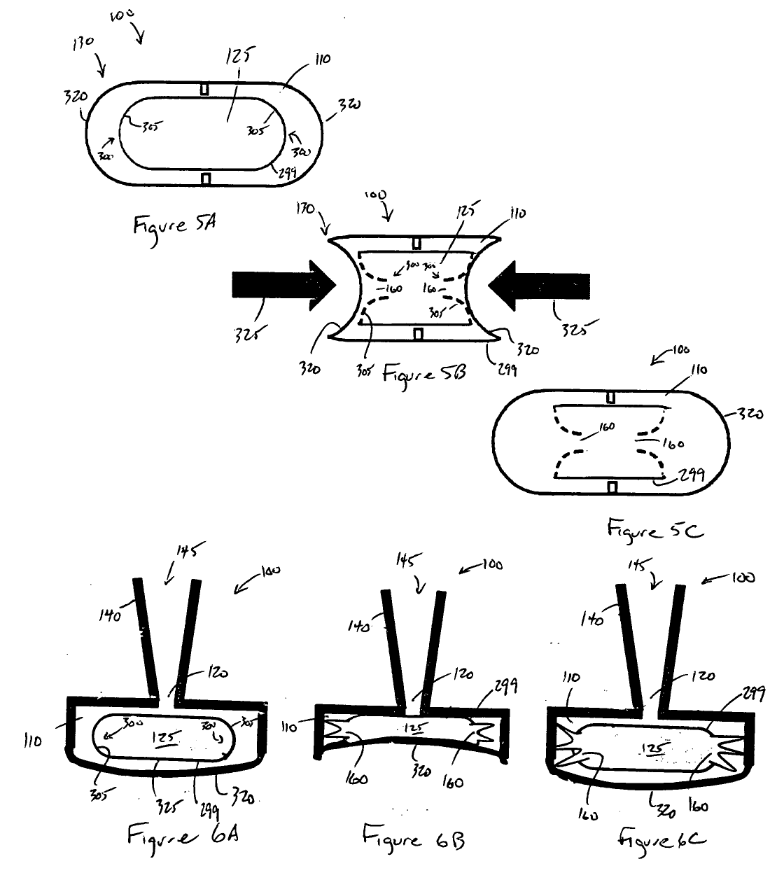

FIGS. 5A through 5C demonstrate another type of an opening mechanism 130 that may be used to create one or more openings in a receptacle 125. In this version, a receptacle 125 having one or more weakened portions 300 is placed in a chamber 110 having one or more flexible sidewalls 320. To create an opening 160 into the receptacle 125, a user applies a force 325 to one or more of the flexible sidewalls 320 to cause a flexible sidewall 320 to contact a weakened portion 300 of the receptacle 125, as shown in FIG. 5B. The force against the weakened portion 300 creates the one or more openings 160 illustrated in FIG. 5C. The flexible sidewall 320 may be biased to its extended position so that it returns to the configuration shown in FIG. 5C when the force 325 is removed. The opening mechanism 130 of this version or of any other version may either be included within the aerosolization apparatus 100 or may be separate from the apparatus so that the receptacle may be opened before, during or after being inserted into the aerosolization apparatus 100.

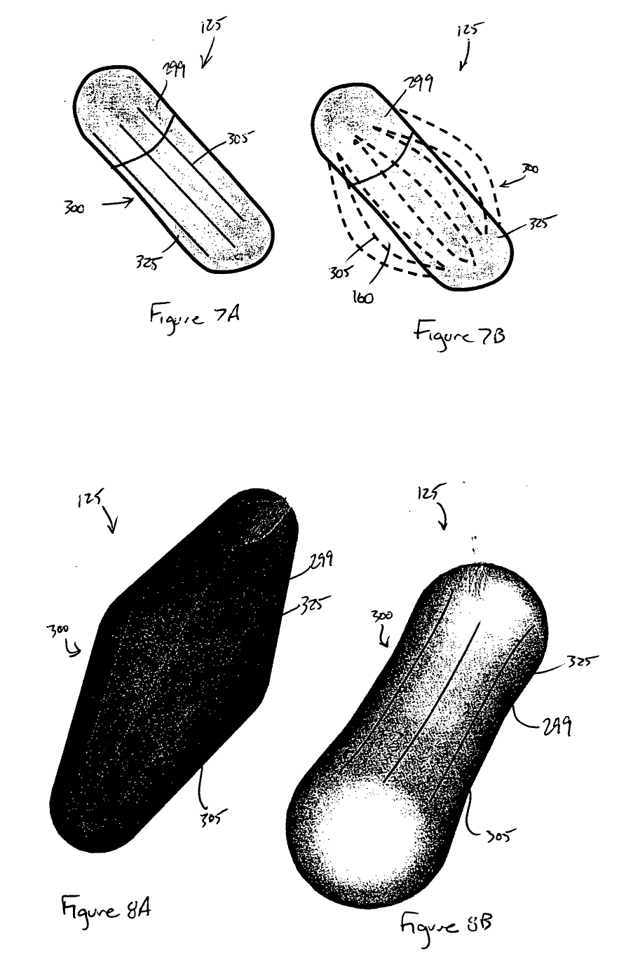

Another version of an aerosolization apparatus 100 having an opening mechanism 130 is shown in FIGS. 6A through 6C. In this version, a flexible wall 320 of the chamber 110 is forced by a user to cause the flexible sidewall 320 to contact a portion of the receptacle 125 other than the weakened portion 300. The stress on the receptacle 125 causes the weakened portion 300 to fracture to create the one or more openings 160. For example, the flexible sidewall 320 may contact an elongated portion 325 of the receptacle 125 to cause a weakened portion 300 in an end of the receptacle 125 to fracture.

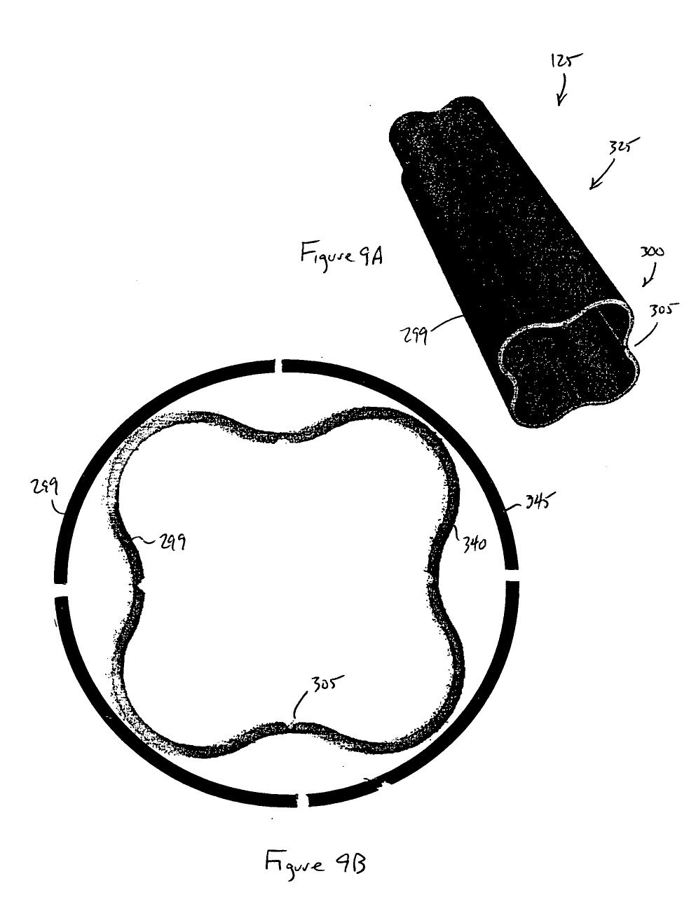

In another version, the weakened portion 300 of a receptacle 125 may be along an elongated portion 325 of the receptacle 125, as shown in FIG. 7A. For example, the elongated portion 325 may comprises one or more weakened lines 305 that extend along the elongated portion 325. When a force is applied to the receptacle 125, the weakened portion 300 opens as shown in FIG. 7B. In one version, the receptacle 125 of FIG. 7A may be inserted into a chamber 110 such as the chamber 110 of the version of FIGS. 5A through 5C. The contacting of the ends of the receptacle 125 causes the openings 160 of FIG. 7B to be created. FIGS. 8A and 8B show different designs of the receptacle 125.

In one version, the receptacle 125 of FIGS. 7A and 7B may be designed to be opened in the absence of an opening mechanism 130. For example, by inserting the receptacle 125 of FIGS. 7A and 7B into a swirling airflow chamber, such as the chamber described above in connection with FIGS. 3A through 3E, the rotation of the receptacle 125 during the aerosolization process generates a sufficient force to cause the receptacle to open to the configuration shown in FIG. 7B. This allows the aerosolization apparatus 100 to be manufactured in a much simpler manner. Similarly, the versions of FIGS. 8A and 8B may be opened without the need for an opening mechanism.

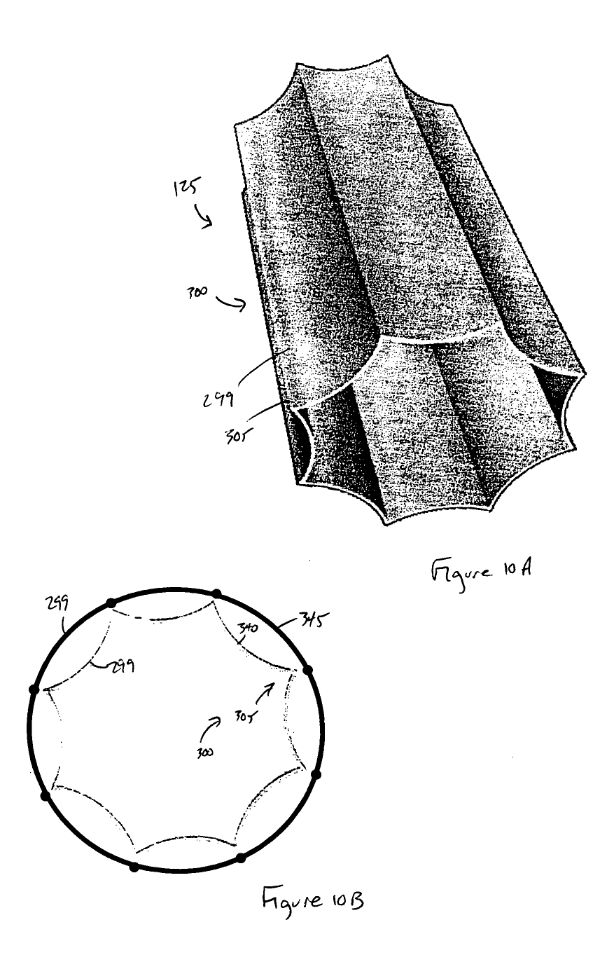

Specific designs of an elongated portion 320 of a receptacle 125 having a weakened portion 300 along the elongate portion 320 are shown in FIGS. 9A and 9B and in FIGS. 10A and 10B. FIGS. 9B and 10B show cross sections through the elongate portions 320 of FIGS. 9A and 10A, respectively, in an unstressed state 340 and in a stressed state 345. These versions are particularly effective when opened by the aerosolization forces instead of an opening mechanism.

In another version, the aerosolization apparatus 100 may be configured differently than as shown in FIGS. 1A through 1E and 3A through 3E. For example, the chamber 100 may be sized and shaped to receive the receptacle 125 so that the receptacle 125 is orthogonal to the inhalation direction, as described in U.S. Pat. No. 3,991,761. As also described in U.S. Pat. No. 3,991,761, the opening mechanism 130 may contact both ends of the receptacle 125. In another version, the chamber may receive the receptacle 125 in a manner where air flows through the receptacle 125 as described for example in U.S. Pat. No. 4,338,931 and in U.S. Pat. No. 5,619,985. In another version, the aerosolization of the pharmaceutical formulation may be accomplished by pressurized gas flowing through the inlets, as described for example in U.S. Pat. No. 5,458,135, U.S. Pat. No. 5,785,049, and U.S. Pat. No. 6,257,233, or propellant, as described in PCT Publication WO 00/72904 and U.S. Pat. No. 4,114,615. All of the above references being incorporated herein by reference in their entireties.

In one version, the receptacle 125 comprises a capsule type receptacle. The capsule may be of a suitable shape, size, and material to contain the pharmaceutical formulation and to provide the pharmaceutical formulation in a usable condition. For example, the capsule may comprise a wall 299 which comprises a material that does not adversely react with the pharmaceutical formulation. In addition, the wall may comprise a material that allows the capsule to be opened to allow the pharmaceutical formulation to be aerosolized. In one version, the wall comprises one or more of gelatin, hydroxypropyl methylcellulose (HPMC), polyethyleneglycol-compounded HPMC, hydroxyproplycellulose, agar, or the like. Alternatively or additionally, the capsule wall may comprise a polymeric material, such as polyvinyl chloride (PVC). In one version, the capsule may comprise telescopically a joined sections, as described for example in U.S. Pat. No. 4,247,066 which is incorporated herein by reference in its entirety. The interior of the capsule may be filled with a suitable amount of the pharmaceutical formulation, and the size of the capsule may be selected to adequately contain a desired amount of the pharmaceutical formulation. The sizes generally range from size 5 to size 000 with the outer diameters ranging from about 4.91 mm to 9.97 mm, the heights ranging from about 11.10 mm to about 26.14 mm, and the volumes ranging from about 0.13 ml to about 1.37 ml, respectively. Suitable capsules are available commercially from, for example, Shionogi Qualicaps Co. in Nara, Japan and Capsugel in Greenwood, S.C. After filling, a top portion may be placed over the bottom portion to form the a capsule shape and to contain the powder within the capsule, as described in U.S. Pat. No. 4,846,876, U.S. Pat. No. 6,357,490, and in the PCT application WO 00/07572 published on Feb. 17, 2000, all of which are incorporated herein by reference in their entireties.

In a preferred version, the invention provides a system and method for aerosolizing a pharmaceutical formulation and delivering the pharmaceutical formulation to the respiratory tract of the user, and in particular to the lungs of the user. The pharmaceutical formulation may comprise powdered medicaments, liquid solutions or suspensions, and the like, and may include an active agent.

The active agent described herein includes an agent, drug, compound, composition of matter or mixture thereof which provides some pharmacologic, often beneficial, effect. This includes foods, food supplements, nutrients, drugs, vaccines, vitamins, and other beneficial agents. As used herein, the terms further include any physiologically or pharmacologically active substance that produces a localized or systemic effect in a patient. An active agent for incorporation in the pharmaceutical formulation described herein may be an inorganic or an organic compound, including, without limitation, drugs which act on: the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system, and the central nervous system. Suitable active agents may be selected from, for example, hypnotics and sedatives, psychic energizers, tranquilizers, respiratory drugs, anticonvulsants, muscle relaxants, antiparkinson agents (dopamine antagnonists), analgesics, anti-inflammatories, antianxiety drugs (anxiolytics), appetite suppressants, antimigraine agents, muscle contractants, anti-infectives (antibiotics, antivirals, antifungals, vaccines) antiarthritics, antimalarials, antiemetics, anepileptics, bronchodilators, cytokines, growth factors, anti-cancer agents, antithrombotic agents, antihypertensives, cardiovascular drugs, antiarrhythmics, antioxicants, anti-asthma agents, hormonal agents including contraceptives, sympathomimetics, diuretics, lipid regulating agents, antiandrogenic agents, antiparasitics, anticoagulants, neoplastics, antineoplastics, hypoglycemics, nutritional agents and supplements, growth supplements, antienteritis agents, vaccines, antibodies, diagnostic agents, and contrasting agents. The active agent, when administered by inhalation, may act locally or systemically.

The active agent may fall into one of a number of structural classes, including but not limited to small molecules, peptides, polypeptides, proteins, polysaccharides, steroids, proteins capable of eliciting physiological effects, nucleotides, oligonucleotides, polynucleotides, fats, electrolytes, and the like.

Examples of active agents suitable for use in this invention include but are not limited to one or more of calcitonin, amphotericin B, erythropoietin (EPO), Factor VIfI, Factor IX, ceredase, cerezyme, cyclosporin, granulocyte colony stimulating factor (GCSF), thrombopoietin (TPO), alpha-1 proteinase inhibitor, elcatonin, granulocyte macrophage colony stimulating factor (GMCSF), growth hormone, human growth hormone (HGH), growth hormone releasing hormone (GHRH), heparin, low molecular weight heparin (LMWH), interferon alpha, interferon beta, interferon gamma, interleukin-1 receptor, interleukin-2, interleukin-1 receptor antagonist, interleukin-3, interleukin-4, interleukin-6, luteinizing hormone releasing hormone (LHRH), factor IX, insulin, pro-insulin, insulin analogues (e.g., mono-acylated insulin as described in U.S. Pat. No. 5,922,675, which is incorporated herein by reference in its entirety), amylin, C-peptide, somatostatin, somatostatin analogs including octreotide, vasopressin, follicle stimulating hormone (FSH), insulin-like growth factor (IGF), insulintropin, macrophage colony stimulating factor (M-CSF), nerve growth factor (NGF), tissue growth factors, keratinocyte growth factor (KGF), glial growth factor (GGF), tumor necrosis factor (TNF), endothelial growth factors, parathyroid hormone (PTH), glucagon-like peptide thymosin alpha 1, IIb/IIIa inhibitor, alpha-1 antitrypsin, phosphodiesterase (PDE) compounds, VLA-4 inhibitors, bisphosponates, respiratory syncytial virus antibody, cystic fibrosis transmembrane regulator (CFTR) gene, deoxyreibonuclease (Dnase), bactericidal/permeability increasing protein (BPI), anti-CMV antibody, 13-cis retinoic acid, macrolides such as erythromycin, oleandomycin, troleandomycin, roxithromycin, clarithromycin, davercin, azithromycin, flurithromycin, dirithromycin, josamycin, spiromycin, midecamycin, leucomycin, miocamycin, rokitamycin, andazithromycin, and swinolide A; fluoroquinolones such as ciprofloxacin, ofloxacin, levofloxacin, trovafloxacin, alatrofloxacin, moxifloxicin, norfloxacin, enoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin, and sitafloxacin, aminoglycosides such as gentamicin, netilmicin, paramecin, tobramycin, amikacin, kanamycin, neomycin, and streptomycin, vancomycin, teicoplanin, rampolanin, mideplanin, colistin, daptomycin, gramicidin, colistimethate, polymixins such as polymixin B, capreomycin, bacitracin, penems; penicillins including penicllinase-sensitive agents like penicillin G, penicillin V, penicillinase-resistant agents like methicillin, oxacillin, cloxacillin, dicloxacillin, floxacillin, nafcillin; gram negative microorganism active agents like ampicillin, amoxicillin, and hetacillin, cillin, and galampicillin; antipseudomonal penicillins like carbenicillin, ticarcillin, azlocillin, mezlocillin, and piperacillin; cephalosporins like cefpodoxime, cefprozil, ceftbuten, ceftizoxime, ceftriaxone, cephalothin, cephapirin, cephalexin, cephradrine, cefoxitin, cefamandole, cefazolin, cephaloridine, cefaclor, cefadroxil, cephaloglycin, cefuroxime, ceforanide, cefotaxime, cefatrizine, cephacetrile, cefepime, cefixime, cefonicid, cefoperazone, cefotetan, cefinetazole, ceftazidime, loracarbef, and moxalactam, monobactams like aztreonam; and carbapenems such as imipenem, meropenem, pentamidine isethiouate, albuterol sulfate, lidocaine, metaproterenol sulfate, beclomethasone diprepionate, triamcinolone acetamide, budesonide acetonide, fluticasone, ipratropium bromide, flunisolide, cromolyn sodium, ergotamine tartrate and where applicable, analogues, agonists, antagonists, inhibitors, and pharmaceutically acceptable salt forms of the above. In reference to peptides and proteins, the invention is intended to encompass synthetic, native, glycosylated, unglycosylated, pegylated forms, and biologically active fragments and analogs thereof.

Active agents for use in the invention further include nucleic acids, as bare nucleic acid molecules, vectors, associated viral particles, plasmid DNA or RNA or other nucleic acid constructions of a type suitable for transfection or transformation of cells, i.e., suitable for gene therapy including antisense. Further, an active agent may comprise live attenuated or killed viruses suitable for use as vaccines. Other useful drugs include those listed within the Physician's Desk Reference (most recent edition).

The amount of active agent in the pharmaceutical formulation will be that amount necessary to deliver a therapeutically effective amount of the active agent per unit dose to achieve the desired result. In practice, this will vary widely depending upon the particular agent, its activity, the severity of the condition to be treated, the patient population, dosing requirements, and the desired therapeutic effect. The composition will generally contain anywhere from about 1% by weight to about 99% by weight active agent, typically from about 2% to about 95% by weight active agent, and more typically from about 5% to 85% by weight active agent, and will also depend upon the relative amounts of additives contained in the composition. The compositions of the invention are particularly useful for active agents that are delivered in doses of from 0.001 mg/day to 100 mg/day, preferably in doses from 0.01 mg/day to 75 mg/day, and more preferably in doses from 0.10 mg/day to 50 mg/day. It is to be understood that more than one active agent may be incorporated into the formulations described herein and that the use of the term “agent” in no way excludes the use of two or more such agents.

The pharmaceutical formulation may comprise a pharmaceutically acceptable excipient or carrier which may be taken into the lungs with no significant adverse toxicological effects to the subject, and particularly to the lungs of the subject. In addition to the active agent, a pharmaceutical formulation may optionally include one or more pharmaceutical excipients which are suitable for pulmonary administration. These excipients, if present, are generally present in the composition in amounts ranging from about 0.01% to about 95% percent by weight, preferably from about 0.5 to about 80%, and more preferably from about 1 to about 60% by weight. Preferably, such excipients will, in part, serve to further improve the features of the active agent composition, for example by providing more efficient and reproducible delivery of the active agent, improving the handling characteristics of powders, such as flowability and consistency, and/or facilitating manufacturing and filling of unit dosage forms. In particular, excipient materials can often function to further improve the physical and chemical stability of the active agent, minimize the residual moisture content and hinder moisture uptake, and to enhance particle size, degree of aggregation, particle surface properties, such as rugosity, ease of inhalation, and the targeting of particles to the lung. One or more excipients may also be provided to serve as bulking agents when it is desired to reduce the concentration of active agent in the formulation.

Pharmaceutical excipients and additives useful in the present pharmaceutical formulation include but are not limited to amino acids, peptides, proteins, non-biological polymers, biological polymers, carbohydrates, such as sugars, derivatized sugars such as alditols, aldonic acids, esterified sugars, and sugar polymers, which may be present singly or in combination. Suitable excipients are those provided in WO 96/32096, which is incorporated herein by reference in its entirety. The excipient may have a glass transition temperature (Tg) above about 35° C., preferably above about 40° C., more preferably above 45° C., most preferably above about 55° C.

Exemplary protein excipients include albumins such as human serum albumin (HSA), recombinant human albumin (rHA), gelatin, casein, hemoglobin, and the like. Suitable amino acids (outside of the dileucyl-peptides of the invention), which may also function in a buffering capacity, include alanine, glycine, arginine, betaine, histidine, glutamic acid, aspartic acid, cysteine, lysine, leucine, isoleucine, valine, methionine, phenylalanine, aspartame, tyrosine, tryptophan, and the like. Preferred are amino acids and polypeptides that function as dispersing agents. Amino acids falling into this category include hydrophobic amino acids such as leucine, valine, isoleucine, tryptophan, alanine, methionine, phenylalanine, tyrosine, histidine, and proline. Dispersibility-enhancing peptide excipients include dimers, trimers, tetramers, and pentamers comprising one or more hydrophobic amino acid components such as those described above.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), pyranosyl sorbitol, myoinositol and the like.

The pharmaceutical formulation may also include a buffer or a pH adjusting agent, typically a salt prepared from an organic acid or base. Representative buffers include organic acid salts of citric acid, ascorbic acid, gluconic acid, carbonic acid, tartaric acid, succinic acid, acetic acid, or phthalic acid, Tris, tromethamine hydrochloride, or phosphate buffers.

The pharmaceutical formulation may also include polymeric excipients/additives, e.g., polyvinylpyrrolidones, derivatized celluloses such as hydroxymethylcellulose, hydroxyethylcellulose, and hydroxypropylmethylcellulose, Ficolls (a polymeric sugar), hydroxyethylstarch, dextrates (e.g., cyclodextrins, such as 2-hydroxypropyl-β-cyclodextrin and sulfobutylether-β-cyclodextrin), polyethylene glycols, and pectin.

The pharmaceutical formulation may further include flavoring agents, taste-masking agents, inorganic salts (for example sodium chloride), antimicrobial agents (for example benzalkonium chloride), sweeteners, antioxidants, antistatic agents, surfactants (for example polysorbates such as “TWEEN 20” and “TWEEN 80”), sorbitan esters, lipids (for example phospholipids such as lecithin and other phosphatidylcholines, phosphatidylethanolamines), fatty acids and fatty esters, steroids (for example cholesterol), and chelating agents (for example EDTA, zinc and other such suitable cations). Other pharmaceutical excipients and/or additives suitable for use in the compositions according to the invention are listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), both of which are incorporated herein by reference in their entireties.

“Mass median diameter” or “MMD” is a measure of mean particle size, since the powders of the invention are generally polydisperse (i.e., consist of a range of particle sizes). MMD values as reported herein are determined by centrifugal sedimentation, although any number of commonly employed techniques can be used for measuring mean particle size. “Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, generally in air, as the particle. The aerodynamic diameter encompasses particle shape, density and physical size of a particle. As used herein, MMAD refers to the midpoint or median of the aerodynamic particle size distribution of an aerosolized powder determined by cascade impaction.

In one version, the powdered formulation for use in the present invention includes a dry powder having a particle size selected to permit penetration into the alveoli of the lungs, that is, preferably 10 μm mass median diameter (MMD), preferably less than 7.5 μm, and most preferably less than 5 μm, and usually being in the range of 0.1 μm to 5 μm in diameter. The delivered dose efficiency (DDE) of these powders may be greater than 30%, more preferably greater than 40%, more preferably greater than 50% and most preferably greater than 60% and the aerosol particle size distribution is about 1.0-5.0 μm mass median aerodynamic diameter (MMAD), usually 1.5-4.5 μm MMAD and preferably 1.5-4.0 μm MMAD. These dry powders have a moisture content below about 10% by weight, usually below about 5% by weight, and preferably below about 3% by weight. Such powders are described in WO 95/24183, WO 96/32149, WO 99/16419, and WO 99/16422, all of which are all incorporated herein by reference in their entireties.

Although the present invention has been described in considerable detail with regard to certain preferred versions thereof, other versions are possible, and alterations, permutations and equivalents of the version shown will become apparent to those skilled in the art upon a reading of the specification and study of the drawings. For example, the cooperating components may be reversed or provided in additional or fewer number. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

1. An aerosolization system comprising:
an aerosolization device comprising a chamber adapted to receive a receptacle; and
a receptacle containing a pharmaceutical formulation, the receptacle comprising a wall having a weakened portion that opens when a force is applied,
whereby an opening into the receptacle may be created at the weakened portion before, during, or after insertion of the receptacle into the chamber by applying a force to the receptacle.

2. An system according to claim 1 wherein the weakened portion comprises a region of the wall altered so as to fracture at a force less than would be necessary without the alteration.

3. A system according to claim 1 wherein the weakened portion comprises a scored region and/or a portion of the wall having a reduced thickness.

4. A system according to claim 1 wherein the aerosolization device comprises a force applying member to apply a force to the weakened portion to create the opening in the receptacle.

5. A system according to claim 4 wherein the force applying member comprises a moveable portion of the chamber.

6. A system according to claim 5 wherein the movable portion is a flexible wall.

7. A system according to claim 4 wherein the force applying member comprises an opening mechanism slidably moveable within the chamber.

8. A system according to claim 7 wherein the opening mechanism comprises a opening member having a blunt tip.

9. A system according to claim 1 wherein the receptacle comprises a capsule.

10. A system according to claim 9 wherein the capsule comprises a wall comprising one or more of gelatin, hydroxypropyl methylcellulose, polyethyleneglycol-compounded hydroxypropyl methylcellulose, hydroxyproplycellulose, and agar.

11. A system according to claim 1 wherein the receptacle contains a powder pharmaceutical formulation.

12. A system according to claim 11 wherein the powder pharmaceutical formulation comprises particles having a mass median diameter less than 10 μm.

13. A system according to claim 11 wherein the powder pharmaceutical formulation has a moisture content below 5% by weight.

14. A method of aerosolizing a pharmaceutical formulation, the method comprising:
providing an aerosolization device comprising a chamber;
providing a receptacle containing a pharmaceutical formulation, the receptacle comprising a wall having a weakened portion that opens when a force is applied;
applying a force to the receptacle to create an opening at the weakened portion;
before, during, or after applying the force to the receptacle, inserting the receptacle into the chamber; and
aerosolizing the pharmaceutical formulation in the chamber.

15. A method according to claim 14 wherein the force is applied by a blunt member.

16. A method according to claim 14 wherein the force is applied after the receptacle is inserted into the chamber.

17. A method according to claim 16 wherein the force is applied by moving a wall of the chamber.

18. A method according to claim 16 wherein the force is applied by sliding a member within the chamber.

19. A method according to claim 14 wherein the applied force causes the weakened portion to break at a scored region.

20. A method according to claim 14 wherein the applied force causes the weakened portion to break at a region of reduced wall thickness.

21. A method according to claim 14 comprising aerosolizing the pharmaceutical formulation by dispersing the pharmaceutical formulation in an air or gas stream.

22. A method according to claim 21 wherein the air or gas stream is generated by a users inhalation.

23. A method according to claim 21 wherein the air or gas stream is from a source of pressurized gas.

24. A method according to claim 14 wherein the receptacle comprises a capsule.

25. A method according to claim 14 wherein the receptacle contains a powder pharmaceutical formulation.

26. A method according to claim 25 wherein the powder pharmaceutical formulation comprises particles having a mass median diameter less than 10 μm.

27. A receptacle for use in an aerosolization device comprising a chamber adapted to receive the receptacle, the receptacle comprising:
a wall having a weakened portion that opens when a force is applied; and
a pharmaceutical formulation within the wall,
whereby an opening may be created at the weakened portion before, during, or after insertion of the receptacle into the chamber by applying a force to the receptacle.

28. A receptacle according to claim 27 wherein the weakened portion comprises a region of the wall altered so as to fracture at a force less than would be necessary without the alteration.

29. A receptacle according to claim 27 wherein the weakened portion comprises a scored region and/or a portion of the wall having a reduced thickness.

30. A receptacle according to claim 27 wherein the weakened portion is opened when a blunt force is applied.

31. A receptacle according to claim 27 wherein the receptacle is a capsule.

32. A receptacle according to claim 31 wherein the capsule comprises a wall comprising one or more of gelatin, hydroxypropyl methylcellulose, polyethyleneglycol-compounded hydroxypropyl methylcellulose, hydroxyproplycellulose, and agar.

33. A receptacle according to claim 27 wherein the receptacle contains a powder pharmaceutical formulation.

34. A receptacle according to claim 33 wherein the powder pharmaceutical formulation comprises particles having a mass median diameter less than 10 μm.

35. A receptacle according to claim 33 wherein the powder pharmaceutical formulation has a moisture content below 5% by weight.
